(19) United States
(12) Patent Application Publication
(10) Pub. No.: US 2024/0376509 A1
(43) Pub. Date: Nov. 14, 2024

(54) Methods of Controlling Cleavage of Formylglycine-Containing Polypeptides

(71) Applicant: R.P. Scherer Technologies, LLC, Carson City, NV (US)

(72) Inventors: Patrick Holder, Oakland, CA (US); Robyn M. Barfield, Emeryville, CA (US); David Rabuka, Kensington, CA (US); Penelope M. Drake, Castro Valley, CA (US); Yun Cheol Kim, Walnut Creek, CA (US); Gregory T. Bleck, Cross Plains, WI (US)

(21) Appl. No.: 18/290,536

(22) PCT Filed: May 25, 2022
(86) PCT No.: PCT/US22/30922

§ 371 Date: Nov. 14, 2023

Related U.S. Application Data
(60) Provisional application No. 63/193,960, filed on May 27, 2021.

(51) Int. Cl. C12P 21/00; A61K 41/00; C07K 1/14; C07K 16/00; C12N 13/00

(52) U.S. Cl. CPC C12P 21/00; A61K 41/0042; C07K 1/14; C07K 16/00; C12N 13/00; C07K 2317/14

(57) ABSTRACT

Methods for reducing cleavage of a protein comprising a formylglycine (fGly) amino acid is provided. Such methods can involve protecting the protein from exposure to visible light having a wavelength of 500 nm or lower. Also provided herein are methods for inducing cleavage of a protein in a target region, the target region comprising an fGly amino acid. The methods may involve exposing the protein to visible light comprising a wavelength of 300 nm-500 nm in the presence of a flavin. Cleavage of the protein may be carried out in the presence of a molecule that is photoactivated to release singlet oxygen species. Cleavage of the protein may be carried out in the presence of a flavin.

### CROSS-REFERENCE TO RELATED APPLICATIONS

This application claims the benefit of U.S. Provisional Application No. 63/193,690, filed May 27, 2021, the disclosure of which is incorporated herein by reference.

### INTRODUCTION

Strategies for modification of proteins for site-specific labeling or site-specific cleavage has been extensively studied due to the importance of such proteins in research and therapeutics. Proteins that include a formylglycine (fGly) amino acid can be labeled by utilizing the aldehyde moiety of fGly amino acid as a chemical handle for site-specific attachment of a moiety of interest. Proteins are often fused with a tag, such as, a protein or peptide that requires cleavage, e.g., to remove a purification tag. Such tags are usually fused to the protein via a cleavable linker sequence.

### SUMMARY

Methods for reducing cleavage of a protein comprising a formylglycine (fGly) amino acid is provided. Such methods can involve protecting the protein from exposure to visible light having a wavelength of 500 nm or lower. Also provided herein are methods for inducing cleavage of a protein in a target region, the target region comprising an fGly amino acid. The methods may involve exposing the protein to visible light comprising a wavelength of 300 nm-500 nm in the presence of a flavin. Cleavage of the protein may be carried out in the presence of a molecule that is photoactivated to release singlet oxygen species. Cleavage of the protein may be carried out in the presence of a flavin.

### BRIEF DESCRIPTION OF THE DRAWINGS

Included in the drawings are the following figures:

FIG. 1. SDS-PAGE of aldehyde-tagged antibody preparations.

FIG. 2. SDS-PAGE of aldehyde-tagged antibody preparations.

FIG. 3. HPLC of an fGly-containing monoclonal antibody exposed to light in either in cell culture media or in 20 mM sodium citrate, 50 mM sodium chloride.

FIG. 4. Summary of mass spectrometric analysis of antibody fragments in fGly-containing protein preparations before and after exposure to light.

FIG. 5. Analysis of effect of Vitamin B12 versus cell culture media on cleavage of fGly-containing antibody.

FIG. 6A. Analysis of effect of riboflavin and light on cleavage of fGly-containing antibody.

FIG. 6B. Analysis of effect of thiamine and light on cleavage of fGly-containing antibody.

FIG. 7A. Analysis of effect of riboflavin and light on cleavage of fGly peptide, ALfGlyTPSRGSLFTGR (SEQ ID NO:1).

FIG. 7B. Analysis of effect of thiamine and light on cleavage of fGly peptide,

(SEQ ID NO: 1)
ALfGlyTPSRGSLFTGR.

FIG. 8A. Riboflavin and light-mediated cleavage of the GPSVFPLfGlyTPSR (SEQ ID NO:2) peptide yields N- and C-terminal fragments that are detected by reverse phase chromatography. S.M.=Starting Material.

FIG. 8B. Mass spectrometric analysis of peptide fragments observed after cleavage of ALfGlyTPSRGSLFTGR (SEQ ID NO:1) and GPSVFPLfGlyTPSR (SEQ ID NO:2) in the presence of riboflavin and light.

FIG. 9A depicts intensity and wavelengths for lamp output, light transmitted through the listed filters, and riboflavin absorption spectrum.

FIG. 9B depicts photoaction spectrum associated with the listed filters and white light.

FIG. 9C. Results for cleavage of GPSVFPLfGlyTPSR (SEQ ID NO:2) peptide incubated with riboflavin and light with or without a bandpass filter and analyzed by HPLC.

FIG. 10. Kinetics of riboflavin-mediated cleavage of fGly-containing proteins.

FIG. 11A. Effect of the ratio of riboflavin to fGly-containing protein on cleavage assessed by varying protein amounts.

FIG. 11B. Effect of the ratio of riboflavin to fGly-containing protein on cleavage assessed by varying riboflavin amounts.

### DETAILED DESCRIPTION OF EMBODIMENTS OF THE INVENTION

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “an aldehyde tag” includes a plurality of such tags and reference to “the polypeptide” includes reference to one or more polypeptides and equivalents thereof known to those skilled in the art, and so forth.

It is further noted that the claims may be drafted to exclude any element which may be optional. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DEFINITIONS

The terms “polypeptide”, “peptide” and “protein” are used interchangeably herein to refer to a polymeric form of amino acids of any length. Unless specifically indicated otherwise, “polypeptide”, “peptide” and “protein” can include genetically coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, proteins which contain at least one N-terminal methionine residue (e.g., to facilitate production in a recombinant bacterial host cell); immunologically tagged proteins; and the like. In certain embodiments, a polypeptide is an antibody.

“Target polypeptide” is used herein to refer to a polypeptide that is to be modified to include an fGly amino acid as described herein. The modification may be subsequently used for attachment of a moiety of interest or cleavage of the polypeptide.

“Target region” as used herein refers to a sequence in a protein at which cleavage of the protein is desired. Target region can include a sulfatase motif.

By “aldehyde tag” or “ald-tag” is meant an amino acid sequence that contains an amino acid sequence derived from a sulfatase motif which has been converted, by action of a formylglycine generating enzyme (FGE) to contain a 2-formylglycine residue (referred to herein as “fGly”). Such a sulfatase motif is referred to herein as an FGE recognition site (FRS). The fGly residue generated by an FGE may also be referred to as a “formylglycine” or “2-formylglycine”. Stated differently, the term “aldehyde tag” is used herein to refer to an amino acid sequence that includes a “converted” sulfatase motif (i.e., a sulfatase motif in which a cysteine or serine residue has been converted to fGly by action of a FGE. A converted sulfatase motif may be produced from an amino acid sequence that includes an “unconverted” sulfatase motif (i.e., a sulfatase motif in which the cysteine or serine residue has not been converted to fGly by an FGE, but is capable of being converted). By “conversion” as used in the context of action of a FGE on a sulfatase motif refers to biochemical modification of a cysteine or serine residue in a sulfatase motif to a formylglycine (fGly) residue (e.g., Cys to fGly, or Ser to fGly). Additional aspects of aldehyde tags and uses thereof in site-specific protein modification are described in U.S. Pat. Nos. 7,985,783 and 8,729,232, the disclosures of each of which are incorporated herein by reference.

By “conversion” as used in the context of action of a formylglycine generating enzyme (FGE) on a sulfatase motif refers to biochemical modification of a cysteine or serine residue in a sulfatase motif to a formylglycine (fGly) residue (e.g., Cys to fGly, or Ser to fGly).

“Native amino acid sequence” or “parent amino acid sequence” are used interchangeably herein in the context of a target polypeptide to refer to the amino acid sequence of the target polypeptide prior to modification to include at least one heterologous FGE recognition site (FRS).

By “genetically-encodable” as used in reference to an amino acid sequence of polypeptide, peptide or protein means that the amino acid sequence is composed of amino acid residues that are capable of production by transcription and translation of a nucleic acid encoding the amino acid sequence, where transcription and/or translation may occur in a cell or in a cell-free in vitro transcription/translation system.

The term “control sequences” refers to DNA sequences to facilitate expression of an operably linked coding sequence in a particular expression system, e.g. mammalian cell, bacterial cell, cell-free synthesis, etc. The control sequences that are suitable for prokaryote systems, for example, include a promoter, optionally an operator sequence, and a ribosome binding site. Eukaryotic cell systems may utilize promoters, polyadenylation signals, and enhancers.

A nucleic acid is “operably linked” when it is placed into a functional relationship with another nucleic acid sequence. For example, a nucleic acid encoding a presequence or secretory leader is operably linked to another nucleic acid encoding a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate the initiation of translation.

The term “expression cassette” as used herein refers to a segment of nucleic acid, usually DNA, that can be inserted into a nucleic acid (e.g., by use of restriction sites compatible with ligation into a construct of interest or by homologous recombination into a construct of interest or into a host cell genome). In general, the nucleic acid segment comprises a polynucleotide that encodes a polypeptide of interest, and the cassette and restriction sites are designed to facilitate insertion of the cassette in the proper reading frame for transcription and translation. Expression cassettes can also comprise elements that facilitate expression of a polynucleotide encoding a polypeptide of interest in a host cell. These elements may include, but are not limited to: a promoter, a minimal promoter, an enhancer, a response element, a terminator sequence, a polyadenylation sequence, and the like.

As used herein the term “isolated” is meant to describe a compound of interest that is in an environment different from that in which the compound naturally occurs. “Isolated” is meant to include compounds that are within samples that are substantially enriched for the compound of interest and/or in which the compound of interest is partially or substantially purified.

As used herein, the term “substantially purified” refers to a compound that is removed from its natural environment and is at least 60% free, usually 75% free, and most usually 90% free from other components with which it is naturally associated.

The term “physiological conditions” is meant to encompass those conditions compatible with living cells, e.g., predominantly aqueous conditions of a temperature, pH, salinity, etc. that are compatible with living cells.

By “heterologous” is meant that a first entity and second entity (or more entities) are provided in an association that is not normally found in nature. For example, a protein containing first sequence and a second sequence where the two sequences do not exist in a single protein in nature.

“N-terminus” refers to the terminal amino acid residue of a polypeptide having a free amine group, which amine group in non-N-terminus amino acid residues normally forms part of the covalent backbone of the polypeptide.

“C-terminus” refers to the terminal amino acid residue of a polypeptide having a free carboxyl group, which carboxyl group in non-C-terminus amino acid residues normally forms part of the covalent backbone of the polypeptide.

By “N-terminal” is meant the region of a polypeptide that is closer to the N-terminus than to the C-terminus.

By “C-terminal” is meant the region of a polypeptide that is closer to the C-terminus than to the N-terminus.

The terms “visible light” and “light” are used herein interchangeably to refer to the segments of the electromagnetic spectrum that the human eye can see. Typically, the healthy human eye can detect wavelengths in the range of about 380 nm to about 700 nm which wavelengths form the visible light spectrum. The visible light spectrum includes six different colors. Red light has a wavelength of about 700 nm to about 620 nm. Orange light has a wavelength of about 620 nm to about 597 nm. Yellow light has a wavelength of about 597 nm to about 577 nm. Green light has a wavelength of about 577 nm to about 492 nm. Blue light has a wavelength of about 492 nm to about 455 nm. Violet light has a wavelength of about 455 nm to about 380 nm.

The term “flavin” as used herein refers to riboflavin and derivatives and analogs thereof, such as, flavin mononucleotide (FMN), flavin adenine dinucleotide (FAD), flavosemiquinone, sulforiboflavin, ester derivatives of riboflavin, riboflavin tetracarboxylate, riboflavin acetic acid, riboflavin tetraacetate, riboflavin propionic acid, roseoflavin, etc. Riboflavin is also known as vitamin B2.

### Methods of Reducing Photo-Clipping of fGly Containing Proteins

A method of reducing cleavage of a protein comprising a formylglycine (fGly) amino acid is provided. The method includes protecting the protein from exposure to visible light having a wavelength of 500 nm or lower.

In certain embodiments, the cleavage of the protein may occur in the presence of a molecule that is photoactivated to release singlet oxygen species, e.g., when the protein is present in a solution that also includes the molecule. In certain embodiments, the molecule is photoactivated by exposure to visible light having a wavelength of 500 nm or lower, e.g., 300 nm-500 nm. In certain embodiments, the cleavage of the protein may occur in the presence of a flavin, e.g., when the protein is present in a solution that also includes the flavin. In certain embodiments, the molecule that is photoactivated to release singlet oxygen species may be a flavin, e.g., a flavin is photoactivated by exposure to visible light having a wavelength of 500 nm or lower, e.g., 300 nm-500 nm. In certain embodiments, the cleavage of the protein may occur in a cell expressing the protein, in a cell culture medium, or both. The cell culture medium may be any standard growth medium used for culturing cells, such as, prokaryotic or eukaryotic cells.

In certain embodiments, the method includes culturing a cell, where the cell expresses the protein and where protecting the protein from exposure to visible light having a wavelength of 500 nm or lower includes using visible light having a wavelength higher than 500 nm during the culturing. For example, the cell culture may be incubated and/or handled in an ambient light that is limited to a wavelength higher than 500 nm, i.e., the ambient light does not include light having a wavelength of 500 nm or lower. In certain embodiments, handling the cell culture may include a step of separating a cell culture medium from the cells which step is performed in light having a wavelength higher than 500 nm. Thus, the visible light to which the protein is exposed during culturing and/or separation of culture medium from cells may be limited to one or more of: red light, green light (e.g., a wavelength of 500 nm to 577 nm), yellow light and orange light. In certain embodiments, the visible light to which the protein is exposed during culturing and/or separation of culture medium from cells may be limited to one or more of: green light, yellow light and orange light, and does not include red light.

In certain embodiments, the cell culture may be placed in an incubator that does not allow substantial amount of visible light to enter the incubator. Such an incubator may be one that is made from an opaque material that is substantially impermeable to light. In addition, the incubator may be housed such that the ambient light has a wavelength higher than 500 nm, where such ambient light protects the protein from cleavage due to exposure to visible light having a wavelength of 500 nm or lower when the incubator door is opened and the cell culture is exposed to ambient light. In certain embodiments, the cell culture may be placed in an incubator that does allow substantial amount of visible light to enter the incubator, e.g., through a glass door. In such an embodiment, the ambient light around the incubator may be limited to light of wavelength higher than 500 nm. In certain embodiments, the cell culture is grown in a container that is impermeable to light having a wavelength of 500 nm or lower, thereby protecting the protein expressed by the cells in the cell culture from exposure to light having a wavelength of 500 nm or lower. In certain embodiments, the method for reducing cleavage of may include culturing the protein in absence of visible light.

In certain embodiments, the method may include synthesizing the protein, and where protecting the protein from exposure to visible light having a wavelength of 500 nm or lower comprises synthesizing the protein in visible light limited to a wavelength higher than 500 nm, i.e., the visible light does not include light having a wavelength of 500 nm or lower. In certain embodiments, the portion of the visible light to which the protein is exposed during synthesis may be limited to one or more of: red light, green light (e.g., a wavelength of 500 nm to 577 nm), yellow light and orange light. In certain embodiments, the portion of the visible light to which the protein is exposed during synthesis may be limited to one or more of: green light (e.g., a wavelength of 500 nm to 577 nm), yellow light and orange light, and does not include red light. In certain embodiments, the method for reducing cleavage may include synthesizing the protein in absence of visible light.

In certain embodiments, the method may include purifying the protein from a cell culture medium, where protecting the protein from exposure to visible light having a wavelength of 500 nm or lower comprises purifying the protein in visible light limited to a wavelength higher than 500 nm. In certain embodiments, the portion of the visible light to which the protein is exposed during purification may be limited to one or more of: red light, green light (e.g., a wavelength of 500 nm to 577 nm), yellow light and orange light. In certain embodiments, the portion of the visible light to which the protein is exposed during purification may be limited to one or more of: green light (e.g., a wavelength of 500 nm to 577 nm), yellow light and orange light, and does not include red light. In certain embodiments, the method for reducing cleavage of may include purifying the protein in absence of visible light. In certain embodiments, purifying the protein may include separating the cells from the cell culture medium, processing the cells if the protein is located in or on the cell or processing the cell culture medium if the protein is secreted. Processing the cells if the protein is located in or on the cell may include lysing the cell.

The visible light that is used for culturing, synthesizing, and/or purifying the protein may be higher than 500 nm, e.g., the visible light having wavelength higher than 500 nm may be visible light having a wavelength higher than 510 nm, higher than 520 nm, higher than 530 nm, higher than 540 nm, higher than 550 nm, or higher, up to about 700 nm. In certain embodiments, the visible light that is used for culturing, synthesizing, and/or purifying the protein may be higher than 500 nm and lower than 620 nm, e.g., the visible light having wavelength higher than 500 nm may be visible light having a wavelength in the range of 510 nm to less 620 nm, 520 nm to less 620 nm, 530 nm to less 620 nm, 540 nm to less 620 nm, or 550 nm to less 620 nm.

In certain embodiments, the method for reducing cleavage of a protein comprising a formylglycine (fGly) amino acid may include exposing the protein to visible light limited to light in the wavelength to 550 nm to 610 nm while avoiding exposure of the protein to light having a wavelength in the range of 500 nm to 380 nm. In certain embodiments, the protein may be present in a solution comprising a molecule that is photoactivated to release singlet oxygen species. In certain embodiments, the protein may be present in a solution comprising a flavin.

In certain embodiments, the visible light having a wavelength higher than 500 nm is generated by passing visible light that comprises light in wavelengths from about 380 nm to about 700 nm (e.g., 400 nm-700 nm) through a filter that significantly blocks transmission of visible light in the range of 380 nm to 500 nm. In certain embodiments, one or more filters may be utilized to block transmission of visible light in the range of 380 nm to 500 nm. The filter or filters may be positioned adjacent a light source that produces light that includes light of 380 nm to 500 nm wavelength. In certain embodiments, the visible light having a wavelength higher than 500 nm is generated by using a light source that produces such light and does not produce light in the wavelength range of 380 nm to 500 nm.

In certain embodiments, the filter or filters may be bandpass filters. The bandpass filter can be an interference filter, and can comprise, for example, distributed Bragg reflectors (DBR) placed in a stacked configuration. Although a DBR can act as a narrow bandwidth reflectors when used individually, when placed in a stacked configuration at close proximity (e.g., at specified distances related to transmission wavelength), DBRs can act as narrow band transmission filters with a high degree of rejection outside of the band. According to an embodiment, the bandpass filter may comprise a material such as gallium arsenide (GaAs), although other materials are able to be used. The DBRs for use as a bandpass filter can be fabricated via deposition of GaAs, as well as other similar materials (e.g., indium gallium arsenide (InGaAs) and others). Doped versions of GaAs with different indices of refraction can produce the required structures for the DBR. The simplest form of bandpass filter has a relatively narrow bandpass (e.g., transmission band), on the order of a few nanometers (nm). However, by using different indices of refraction between the two DBRs, or by varying the thicknesses of layers of the DBRs, the bandwidth can be tuned to be substantially wider than this (e.g., tens to hundreds of nm).

The light source includes, without limitation, an LED lamp, an incandescent lamp, a fluorescent lamp, and a laser. In case of laser or LED, a filter may not be needed and instead the output may be in the desired wavelength range. For example, one or more of a green LED (e.g., a wavelength of 500 nm to 577 nm), yellow LED, orange LED, or red LED may be used as a light source to protect the protein photo-clipping. In certain embodiments, the photo-clipping may be mediated by a molecule that is photoactivated to release singlet oxygen species. In certain embodiments, the photo-clipping may be mediated by a flavin.

In certain embodiments, the molecule that is photoactivated to release singlet oxygen species may be a flavin. In certain embodiments, the flavin may be riboflavin, FMN, or FAD. In certain embodiments, the flavin may be flavosemiquinone, sulforiboflavin, ester derivatives of riboflavin, riboflavin tetracarboxylate, riboflavin acetic acid, riboflavin tetraacetate, riboflavin propionic acid, or roseoflavin.

The protein that includes an fGly residue can be any protein that is modified to include an fGly residue. This protein is also referred to herein as the target protein. The target protein may include more than one fGly residue, e.g., at least 2, 3, 4, 5, 6, or up to 10 fGly residues or more. In some embodiments, fGly residue(s) is introduced using chemical synthesis. In other embodiments, the target protein may be a protein in which an fGly residue is present as a result of action of a FGE on a cysteine or serine residue present in a FGE recognition site. The FGE recognition site is also referred to herein as a sulfatase motif.

In certain embodiments, the fGly residue(s) is located in the target polypeptide at a position that does not adversely affect protein conformation. In some embodiments, it is desirable to position the FGE recognition sites (FRSs) in the target polypeptide taking into account its structure when folded (e.g., in a cell-free environment, usually a cell-free physiological environment) and/or presented in or on a cell membrane (e.g., for cell-membrane associated polypeptides, such as transmembrane proteins). For example, an FRS can be positioned at a solvent accessible site in the folded target polypeptide. The solvent accessible FRS in a folded polypeptide is thus accessible to a FGE for conversion of the serine or cysteine to an fGly. Likewise, a solvent accessible fGly residue in an aldehyde tagged polypeptide is accessible to a reactive partner reagent for conjugation to a moiety of interest. Where an FRS is positioned at a solvent accessible site, in vitro FGE-mediated conversion and conjugation with a moiety by reaction with a reactive partner can be performed without the need to denature the protein. Solvent accessible sites can also include target polypeptide regions that are exposed at an extracellular or intracellular cell surface when expressed in a host cell (e.g., other than a transmembrane region of the target polypeptide).

Accordingly, one or more FRSs can be provided at sites independently selected from, for example, a solvent accessible N-terminus, a solvent accessible N-terminal region, a solvent accessible C-terminus, a solvent accessible C-terminal region, and/or a loop structure (e.g., an extracellular loop structure and/or an intracellular loop structure). In some embodiments, the FRS is positioned at a site other than the C-terminus of the polypeptide. In other embodiments, the polypeptide in which the FRS is positioned is a full-length polypeptide.

In other embodiments, an FRS is positioned at a site which is post-translationally modified in the native target polypeptide. For example, an FRS can be introduced at a site of glycosylation (e.g., N-glycosylation, O-glycosylation), phosphorylation, sulfatation, ubiquitination, acylation, methylation, prenylation, hydroxylation, carboxylation, and the like in the native target polypeptide. Consensus sequences of a variety of post-translationally modified sites, and methods for identification of a post-translationally modified site in a polypeptide, are well known in the art. It is understood that the site of post-translational modification can be naturally-occurring or such a site of a polypeptide that has been engineered (e.g., through recombinant techniques) to include a post-translational modification site that is non-native to the polypeptide (e.g., as in a glycosylation site of a hyperglycosylated variant of EPO). In the latter embodiment, polypeptides that have a non-native post-translational modification site and which have been demonstrated to exhibit a biological activity of interest are of particular interest.

An FRS can be provided in a target polypeptide by insertion (e.g., so as to provide a 5 or 6 amino acid residue insertion within the native amino acid sequence) or by addition (e.g., at an N- or C-terminus of the target polypeptide). An FRS can also be provided by complete or partial substitution of native amino acid residues with the contiguous amino acid sequence of an FRS. For example, a heterologous FRS can be provided in a target polypeptide by replacing 1, 2, 3, 4, or 5 (or 1, 2, 3, 4, 5, or 6) amino acid residues of the native amino acid sequence with the corresponding amino acid residues of the FRS. Target polypeptides having more than one FRSs can be used to provide for attachment of the same moiety or of different moieties at the fGly of the aldehyde tag.

The target polypeptide may be any protein or peptide, e.g., a recombinant protein or peptide. The target polypeptides may be fusion proteins, antibodies (IgG1, 2, 3, 4, IgM, IgA), enzymes (e.g., proteases), hormones, growth factors, receptors, ligands, glycoproteins, a cell signaling protein, and the like, or any combination thereof. Examples of target proteins include cytokines may be an interferon (e.g., IFN-γ, etc.), a chemokine, an interleukin (e.g., IL-2, IL-4, IL-5, IL-6, IL-10, IL-12, IL-17 etc.), a lymphokine, a tumor necrosis factor (e.g., TNF-α, etc.), transforming growth factor β (TGFβ), and the like. In one embodiment, the target polypeptide may provide for a therapeutic benefit, particularly those polypeptides for which attachment to a moiety can provide for one or more of, for example, targeted drug delivery, an increase in serum half-life, a decrease in an adverse immune response, additional or alternate biological activity or functionality, and the like, or other benefit or reduction of an adverse side effect. Where the therapeutic polypeptide is an antigen for a vaccine, modification can provide for an enhanced immunogenicity of the polypeptide.

Examples of classes of therapeutic proteins include those that are cytokines, chemokines, growth factors, hormones, antibodies, and antigens. Further examples include erythropoietin, human growth hormone (hGH), bovine growth hormone (bGH), follicle stimulating hormone (FSH), interferon (e.g., IFN-gamma, IFN-beta, IFN-alpha, IFN-omega, consensus interferon, and the like), insulin, insulin-like growth factor (e.g., IGF-I, IGF-II), blood factors (e.g., Factor VIII, Factor IX, Factor X, tissue plasminogen activator (TPA), and the like), colony stimulating factors (e.g., granulocyte-CSF (G-CSF), macrophage-CSF (M-CSF), granulocyte-macrophage-CSF (GM-CSF), and the like), transforming growth factors (e.g., TGF-beta, TGF-alpha), interleukins (e.g., IL-1, IL-2, IL-3, IL-4, IL-5, IL-6, IL-7, IL-8, IL-12, and the like), epidermal growth factor (EGF), platelet-derived growth factor (PDGF), fibroblast growth factors (FGFs, e.g., aFGF, bFGF), glial cell line-derived growth factor (GDNF), nerve growth factor (NGF), RANTES, and the like.

Further examples include antibodies, e.g., polyclonal antibodies, monoclonal antibodies, humanized antibodies, antigen-binding fragments (e.g., F(ab)′, Fab, Fv), single chain antibodies, IgG (e.g., IgG1, IgG2, IgG3, or IgG4), IgM, IgA, and the like. Of particular interest are antibodies that specifically bind to a tumor antigen, an immune cell antigen (e.g., CD4, CD8, and the like), an antigen of a microorganism, particularly a pathogenic microorganism (e.g., a bacterial, viral, fungal, or parasitic antigen), and the like. Moieties of interest that may be attached using the fGly residue(s) include drugs (e.g., small molecules), polymers (e.g., PEG), detectable labels, etc.

As explained in the working examples, the riboflavin present in the cell culture media causes cleavage of the fGly protein expressed by the cell when exposed to visible light, especially, light having wavelengths in the range of 380-500 nm which is absorbed by the riboflavin. Thus, in the methods disclosed herein the target protein is protected from cleavage by limiting exposure to light having wavelengths in the range of 380-500 nm at least until the protein is separated from riboflavin or a derivative or analog thereof.

### Methods of Inducing Photo-Clipping of fGly Containing Proteins

A method of inducing cleavage of a protein in a target region, where the target region includes a formylglycine (fGly) amino acid is disclosed. The method includes exposing the protein to light comprising a wavelength of 300 nm-500 nm. In certain embodiments, the method may include exposing the protein to light having a wavelength of 325 nm-500 nm, 325 nm-495 nm, 350 nm-500 nm, 350 nm-480 nm, or 350 nm-450 nm. In certain embodiments, the light is limited to wavelength of 300 nm-500 nm and does not include light in the wavelength higher than 500 nm, e.g., 510 nm-700 nm.

In certain embodiments, the protein may be present in a cell culture medium, e.g., a standard growth medium used for culturing prokaryotic cells such as E. coli or a standard growth medium used for culturing eukaryotic cells such as mammalian cells. In certain embodiments, the protein may be present in a solution comprising a molecule that is photoactivated to release singlet oxygen species. In certain embodiments, the molecule is a photosensitizer, such as, porphyrins and their tetrapyrrole analogs such as chlorine, porphycene, phthalocyanine and naphthalocyanine. As used herein, the term photosensitizer refers to a molecule that is photoactivated by absorption of visible light and releases singlet oxygen species. In certain embodiments, the molecule absorbs visible light in the wavelength of 380 nm-500 nm. In certain embodiments, the protein may be present in a solution comprising a flavin. In certain embodiments, the molecule that is photoactivated to release singlet oxygen species may be a flavin. In certain embodiments, a method of inducing cleavage of a protein in a target region, where the target region includes an fGly amino acid may involve exposing a solution comprising the protein and a photosensitizer to visible light. The photosensitizer may be any molecule that is photoactivated by absorption of visible light and releases singlet oxygen species. As explained in the Examples section, riboflavin is a photosensitizer that mediate cleavage of fGly containing proteins when exposed to visible light.

The length of exposure and/or the amount of the molecule (e.g., a flavin) may be varied and can be determined empirically. The time period for which the protein is exposed to the light can also be varied by increasing intensity of the light and/or concentration of the molecule (e.g., a flavin) and/or temperature. In certain embodiments, a solution containing the protein and the molecule (e.g., a flavin) may be exposed to light comprising a wavelength of 300 nm-500 nm for a period of time of 1 minute-48 hours, 3 minutes-40 hours, 5 minutes-36 hours, 10 minutes-24 hours, 15 minutes-20 hours, 20 minutes-10 hours, 1 minute-1 hr, 3 minutes-30 minutes, 1 minute-30 minutes, 5 minutes-30 minutes, or 10 minutes-30 minutes. The concentration of the molecule (e.g., a flavin, such as, riboflavin) in the solution may be at least 0.001 μM, 0.01 μM, 0.03 μM, 0.1 μM, 0.3 μM, 1 μM, 3 μM, 5 μM, 10 μM, or more, e.g., up to 20 μM. The solution comprising the protein and the molecule (e.g., a flavin) may be incubated at 4° C., room temperature, 37° C., or a higher temperature, e.g., up to 60° C., or any temperature between about 4° C. and 60° C., when exposing the protein to light for inducing photo-clipping at fGly residue.

In certain embodiments, the protein may be exposed to visible light in the presence of the molecule (e.g., a flavin) by using any suitable light source, such as, an LED lamp, an incandescent lamp, a fluorescent lamp, or a laser. For example, a violet LED, a blue LED, or a violet and blue LED may be used to induce flavin-mediated (e.g. riboflavin-mediated) photo-clipping of the protein.

The solution containing the protein and the molecule (e.g., a flavin) to be exposed to light may be a solution comprising a buffer, e.g., a buffer having a pH of 7-8, e.g. about 7.4. In certain embodiments, the protein may be protein expressed by cells, and in certain embodiments, secreted into the cell culture medium from cells expressing the protein. In such an embodiment, a molecule that is photoactivated to release singlet oxygen species (e.g., a flavin) present in the cell culture medium may be sufficient to induce cleavage and addition of the isolated molecule (e.g., a flavin) is not needed. In certain embodiments, the flavin present in the cell culture medium may be supplemented by adding flavin to the medium.

In certain embodiments, the flavin may be riboflavin, FMN, or FAD. In certain embodiments, the flavin may be flavosemiquinone, sulforiboflavin, ester derivatives of riboflavin, riboflavin tetracarboxylate, riboflavin acetic acid, riboflavin tetraacetate, riboflavin propionic acid, or roseoflavin.

In certain embodiments, the method may include a step of introducing a formylglycine-generating enzyme (FGE) recognition site in the target region of the protein. The protein that includes an fGly residue can be any protein that is to be reacted by causing cleavage adjacent the fGly residue. This protein is also referred to herein as the target protein. In certain aspects, the target protein may be a protein that includes a secretion signal (e.g., a signal peptide). The secretion signal may be present at the N-terminus of the protein and an fGly residue may be included in a target region located between the secretion signal and the N-terminus of the protein of the rest of the protein. Upon exposure of the secreted protein in the cell culture medium (which includes a flavin, e.g., riboflavin) to visible light, e.g., light having a wavelength 300 nm-500 nm, the secretion signal may be cleaved off.

In another embodiment, the protein may include a tag, e.g., a purification tag at the N-terminus or the C-terminus. An fGly residue may be present in a target site located between the tag and the N-terminus or C-terminus of the protein of the rest of the protein. After the protein has been purified, the tag may be cleaved off by the disclosed method. In some embodiments, the culture and/or purification of the protein may be performed as disclosed in the preceding section to prevent flavin-mediated photo-clipping of the protein. Once the protein has been purified, the purification tag can be removed by inducing photo-clipping by exposure to visible light (e.g., light comprising a wavelength of 300 nm-500 nm) in the presence of a flavin.

The protein that is to be cleaved using the subject method may be any protein, such as, therapeutic proteins described in the preceding section.

In another embodiment, the protein is an antibody comprising an Fc region and the target region is located between the Fc region and a CH1 domain of the antibody. Cleaving the fGly residue in the target region results in generation of Fab and Fc fragments.

In another embodiment, the protein may be associated with the cell membrane of a cell expressing the protein. The protein may include a transmembrane region. The fGly residue may be located in a target region that is N-terminus to the transmembrane region or C-terminus to the transmembrane region. The protein may be attached to the membrane via an anchoring moiety, e.g., a lipid moiety. The fGly residue may be located in a target region that is at or adjacent the C-terminus of the protein prior to the attachment region of the anchoring moiety. Cleavage at the target site using the methods disclosed herein may be used to release the membrane associated protein from the cell surface.

### FGE Recognition Site (FRS)

As noted in the preceding section, one or more fGly residues can be present in the target protein. The one or more fGly residues can be introduced by using chemical synthesis. In other embodiments, the one or more fGly residues are generated by action of an FGE on the sulfatase motif which leads to oxidation of the cysteine or serine in the motif to generate the fGly residue. As used herein, the terms “sulfatase motif” and “FGE recognition site” are used interchangeably and refer to a contiguous sequence of amino acids that is recognized by a FGE. In certain embodiments, a target protein may naturally include a sulfatase motif. In certain embodiments, a target protein may be modified to include a sulfatase motif. A sulfatase motif that is present at a location in a protein where cleavage is desired, in certain embodiments, may be the target site or may be located within a target site.

Any sulfatase motif sequence can be included in the target protein. In some embodiments, a FGE that recognizes the sulfatase motif is either produced by the cells expressing the target protein or is added to the cell culture medium or to the purified protein to convert the C or S residue in the sulfatase motif to fGly.

The FGE may be a eukaryotic FGE (e.g., a mammalian FGE, including a human FGE) or a prokaryotic FGE. The FGE may be a modified FGE such that the modified FGE recognizes different or additional sulfatase motif as compared to the wild-type FGE from which the modified FGE is derived.

The sulfatase motif may have the formula:

X₁(C/S)X₂(P/A)X₂Z₃

where
X₁ may be present or absent and, when present, can be any amino acid, though usually an aliphatic amino acid, a sulfur-containing amino acid, or a polar, uncharged amino acid, (i.e., other than an aromatic amino acid or a charged amino acid), usually L, M, S or V, with the proviso that when the sulfatase motif is at the N-terminus of the target polypeptide, X₁ is present;
X₂ and X₃ independently can be any amino acid, though usually an aliphatic amino acid, a sulfur-containing amino acid, or a polar, uncharged amino acid, (i.e., other than an aromatic amino acid or a charged amino acid), usually S, T, A, V, G, or C, more usually S, T, A, V or G; and
Z₃ is a basic amino acid (which may be other than arginine (R), and may be lysine (K) or histidine (H), usually lysine), or an aliphatic amino acid (alanine (A), glycine (G), leucine (L), valine (V), isoleucine (I), or proline (P), usually A, G, L, V, or I.

An example of a sulfatase motif includes the consensus sequence:

X₁SX₂PX₂R

Another example of sulfatase motif includes the consensus sequence:

X₁CX₂PX₂R

Specific examples of sulfatase motifs include LCTPSR, MCTPSR, VCTPSR, LCSPSR, LCAPSR LCVPSR, LCGPSR, ICTPAR, LCTPSK, MCTPSK, VCTPSK, LCSPSK, LCAPSK, LCVPSK, LCGPSK, LCTPSA, ICTPAA, MCTPSA, VCTPSA, LCSPSA, LCAPSA, LCVPSA, LCGPSA, LSTPSR, LCTASR, and LCTASA. Other specific sulfatase motifs are readily apparent from the disclosure provided herein. A target protein may include one or more of such sulfatase motifs.

### Modification of a Target Polypeptide to Include FGE Recognition Site

Modification of a target polypeptide to include one or more FGE recognition sites can be accomplished using recombinant molecular genetic techniques, so as produce nucleic acid encoding the desired target polypeptide. Such methods are well known in the art, and include cloning methods, site-specific mutation methods, and the like (see, e.g., Sambrook et al., In “Molecular Cloning: A Laboratory Manual” (Cold Spring Harbor Laboratory Press 1989); “Current Protocols in Molecular Biology” (eds., Ausubel et al.; Greene Publishing Associates, Inc., and John Wiley & Sons, Inc. 1990 and supplements). Alternatively, one or more FGE recognition sites can be added using non-recombinant techniques, e.g., using native chemical ligation or pseudo-native chemical ligation, e.g., to add one or more FGE recognition sites to a C-terminus of the target polypeptide (see, e.g., U.S. Pat. Nos. 6,184,344; 6,307,018; 6,451,543; 6,570,040; US 2006/0173159; US 2006/0149039). See also Rush et al. (Jan. 5, 2006) Org Lett. 8(1):131-4.

### Formylglycine Generating Enzymes (FGEs)

Any enzyme that oxidizes cysteine or serine in a sulfatase motif to fGly is referred to herein as a “formylglycine generating enzyme” or “FGE”. Thus, as discussed above, an “FGE” is used herein to refer to any enzyme that can act as an fGly-generating enzyme to mediate conversion of a cysteine (C) of a sulfatase motif to fGly or that can mediate conversion of serine (S) of a sulfatase motif to fGly. It should be noted that in general, the literature refers to fGly-generating enzymes that convert a C to fGly in a sulfatase motif as FGEs, and refers to enzymes that convert S to fGly in a sulfatase motif as Ats-B-like. However, for purposes of the present disclosure “FGE” is used generically to refer to any type of enzyme that exhibits an fGly-generating enzyme activity at a sulfatase motif, with the understanding that an appropriate FGE will be selected according to the target reactive partner containing the appropriate sulfatase motif (i.e., C-containing or S-containing).

As evidenced by the ubiquitous presence of sulfatases having an fGly at the active site, FGEs are found in a wide variety of cell types, including both eukaryotes and prokaryotes. There are at least two forms of FGEs. Eukaryotic sulfatases contain a cysteine in their sulfatase motif and are modified by the “SUMF1-type” FGE (Cosma et al. Cell 2003, 113, (4), 445-56; Dierks et al. Cell 2003, 113, (4), 435-44). The fGly-generating enzyme (FGE) is encoded by the SUMF1 gene. Prokaryotic sulfatases can contain either a cysteine or a serine in their sulfatase motif and are modified either by the “SUMF1-type” FGE or the “AtsB-type” FGE, respectively (Szameit et al. J Biol Chem 1999, 274, (22), 15375-81). In eukaryotes, it is believed that this modification happens co-translationally or shortly after translation in the endoplasmic reticulum (ER) (Dierks et al. Proc Natl Acad Sci USA 1997, 94(22):11963-8). Without being held to theory, in prokaryotes it is thought that SUMF1-type FGE functions in the cytosol and AtsB-type FGE functions near or at the cell membrane. A SUMF2 FGE has also been described in deuterostomia, including vertebrates and echinodermata (see, e.g., Pepe et al. (2003) Cell 113, 445-456, Dierks et al. (2003) Cell 113, 435-444; Cosma et al. (2004) Hum. Mutat. 23, 576-581).

In general, the FGE used to facilitate conversion of cysteine or serine to fGly in a sulfatase motif of in a target polypeptide is selected according to the sulfatase motif present in the target polypeptide. The FGE can be native to the host cell in which the target polypeptide is expressed, or the host cell can be genetically modified to express an appropriate FGE. In some embodiments it may be desired to use a sulfatase motif compatible with a human FGE (e.g., the SUMF1-type FGE, see, e.g., Cosma et al. Cell 113, 445-56 (2003); Dierks et al. Cell 113, 435-44 (2003)), and express the target protein in a human cell that expresses the FGE or in a host cell, usually a mammalian cell, genetically modified to express a human FGE.

In general, an FGE for use in the methods disclosed herein can be obtained from naturally occurring sources or synthetically produced. For example, an appropriate FGE can be derived from biological sources which naturally produce an FGE or which are genetically modified to express a recombinant gene encoding an FGE. Nucleic acids encoding a number of FGEs are known in the art and readily available (see, e.g., Preusser et al. 2005 J. Biol. Chem. 280(15):14900-10 (Epub 2005 Jan. 18); Fang et al. 2004 J Biol Chem. 79(15):14570-8 (Epub 2004 Jan. 28); Landgrebe et al. Gene. 2003 Oct. 16; 316:47-56; Dierks et al. 1998 FEBS Lett. 423(1):61-5; Dierks et al. Cell. 2003 May 16; 113(4):435-44; Cosma et al. (2003 May 16) Cell 113(4):445-56; Baenziger (2003 May 16) Cell 113(4):421-2 (review); Dierks et al. Cell. 2005 May 20; 121(4):541-52; Roeser et al. (2006 Jan. 3) Proc Natl Acad Sci USA 103(1):81-6; Sardiello et al. (2005 Nov. 1) Hum Mol Genet. 14(21):3203-17; WO 2004/072275; and GenBank Accession No. NM_182760. Accordingly, the disclosure here provides for recombinant host cells genetically modified to express an FGE that is compatible for use with the FRS present in the target polypeptide. In one embodiment, an FGE is obtained from Mycobacterium tuberculosis (Mtb), an exemplary Mtb FGE is one having the amino acid sequence provide at GenBank Acc. No. NP_215226 (gi:15607852).

Where a cell-free method is used to convert a sulfatase motif-containing polypeptide, an isolated FGE can be used. Any convenient protein purification procedures may be used to isolate an FGE, see, e.g., Guide to Protein Purification, (Deuthser ed.) (Academic Press, 1990). For example, a lysate may be prepared from a cell that produces a desired FGE, and the FGE purified, e.g., using HPLC, exclusion chromatography, gel electrophoresis, affinity chromatography, and the like.

### Expression Vectors and Host Cells for Production of fGly Polypeptides

The disclosure provides a nucleic acid encoding target polypeptides comprising an FRS or an FRS, as well as constructs and host cells containing the nucleic acid. Such nucleic acids comprise a sequence of DNA having an open reading frame that encodes an FRS or a target polypeptide comprising an FRS and, in most embodiments, is capable, under appropriate conditions, of being expressed. “Nucleic acid” encompasses DNA, cDNA, mRNA, and vectors comprising such nucleic acids.

Nucleic acids encoding an FRS, as well as target polypeptides comprising an FRS, are provided herein. Such nucleic acids include genomic DNAs modified by insertion of an FGE recognition site-encoding sequence and cDNAs encoding the target polypeptides. The term “cDNA” as used herein is intended to include all nucleic acids that share the arrangement of sequence elements found in a native mature mRNA species (including splice variants), where sequence elements are exons and 3′ and 5′ non-coding regions. Normally mRNA species have contiguous exons, with the intervening introns, when present, being removed by nuclear RNA splicing, to create a continuous open reading frame encoding a protein according to the subject invention.

The term “gene” intends a nucleic acid having an open reading frame encoding a polypeptide (e.g., a polypeptide comprising an FGE recognition site), and, optionally, any introns, and can further include adjacent 5′ and 3′ non-coding nucleotide sequences involved in the regulation of expression (e.g., regulators of transcription and/or translation, e.g., promoters, enhancers, translational regulatory signals, and the like), up to about 20 kb beyond the coding region, but possibly further in either direction, which adjacent 5′ and 3′ non-coding nucleotide sequences may be endogenous or heterologous to the coding sequence. Transcriptional and translational regulatory sequences, such as promoters, enhancers, etc., may be included. including about 1 kb, but possibly more, of flanking genomic DNA at either the 5′ or 3′ end of the transcribed region.

Nucleic acids contemplated herein can be provided as part of a vector (also referred to as a construct), a wide variety of which are known in the art and need not be elaborated upon herein. Exemplary vectors include, but are not limited to, plasmids; cosmids; viral vectors (e.g., retroviral vectors); non-viral vectors; artificial chromosomes (YAC's, BAC's, etc.); mini-chromosomes; and the like.

The choice of vector will depend upon a variety of factors such as the type of cell in which propagation is desired and the purpose of propagation. Certain vectors are useful for amplifying and making large amounts of the desired DNA sequence. Other vectors are suitable for expression in cells in culture. Still other vectors are suitable for transfer and expression in cells in a whole animal. The choice of appropriate vector is well within the skill of the art. Many such vectors are available commercially.

To prepare the constructs, a polynucleotide is inserted into a vector, typically by means of DNA ligase attachment to a cleaved restriction enzyme site in the vector. Alternatively, the desired nucleotide sequence can be inserted by homologous recombination or site-specific recombination.

Vectors can provide for extrachromosomal maintenance in a host cell or can provide for integration into the host cell genome. Vectors are amply described in numerous publications well known to those in the art. Exemplary vectors that may be used include but are not limited to those derived from recombinant bacteriophage DNA, plasmid DNA or cosmid DNA. For example, plasmid vectors such as pBR322, pUC 19/18, pUC 118, 119 and the M13 mp series of vectors may be used. Bacteriophage vectors may include λgt10, λgt11, λgt18-23, λZAP/R and the EMBL series of bacteriophage vectors. Cosmid vectors that may be utilized include, but are not limited to, pJB8, pCV 103, pCV 107, pCV 108, pTM, pMCS, pNNL, pHSG274, COS202, COS203, pWE15, pWE16 and the charomid 9 series of vectors. Alternatively, recombinant virus vectors may be engineered, including but not limited to those derived from viruses such as herpes virus, retroviruses, vaccinia virus, poxviruses, adenoviruses, adeno-associated viruses or bovine papilloma virus.

For expression of a polypeptide of interest, an expression cassette may be employed. Thus, the present invention provides a recombinant expression vector comprising a subject nucleic acid. The expression vector provides a transcriptional and translational regulatory sequences, and may provide for inducible or constitutive expression, where the coding region is operably linked under the transcriptional control of the transcriptional initiation region, and a transcriptional and translational termination region. These control regions may be native to the gene encoding the polypeptide (e.g., the target polypeptide or the FGE), or may be derived from exogenous sources. In general, the transcriptional and translational regulatory sequences may include, but are not limited to, promoter sequences, ribosomal binding sites, transcriptional start and stop sequences, translational start and stop sequences, and enhancer or activator sequences.

Expression vectors generally have convenient restriction sites located near the promoter sequence to provide for the insertion of nucleic acid sequences encoding proteins of interest. A selectable marker operative in the expression host may be present to facilitate selection of cells containing the vector. Selection genes are well known in the art and will vary with the host cell used.

An FGE recognition site (FRS)-encoding cassette is also provided herein, which includes a nucleic acid encoding the FRS, and suitable restriction sites flanking the tag-encoding sequence for in-frame insertion of a nucleic acid encoding a target polypeptide. Such an expression construct can provide for addition of an FRS at the N-terminus or C-terminus of a target polypeptide. The FRS cassette can be operably linked to a promoter sequence to provide for expression of the resulting polypeptide comprising the FRS, and may further include one or more selectable markers.

The present disclosure also provides expression cassettes for production of polypeptides comprising an FRS (e.g., having an FRS positioned at a N-terminus, at a C-terminus). Such expression cassettes generally include a first nucleic acid comprising an FRS-encoding sequence, and at least one restriction site for insertion of a second nucleic acid encoding a polypeptide of interest. The restriction sites can be positioned 5′ and/or 3′ of the FRS-encoding sequence. Insertion of the polypeptide-encoding sequence in-frame with the FRS-encoding sequence provides for production of a recombinant nucleic acid encoding a fusion protein that is an FRS containing polypeptide as described herein. Constructs containing such an expression cassette generally also include a promoter operably linked to the expression cassette to provide for expression of the FRS containing polypeptide produced. Other components of the expression construction can include selectable markers and other suitable elements.

### Host Cells

Any of a number of suitable host cells can be used in the production of an FRS containing polypeptide. The host cell used for production of an FRS containing-polypeptide can optionally provide for FGE-mediated conversion (e.g., by action of an FGE native to the host cell (which may be expressed from an endogenous coding sequence in the cell and/or produced from a recombinant construct), by action of an FGE that is not native to the host cell, or both), so that the polypeptide produced contains an aldehyde tag following expression and post-translational modification by FGE. Alternatively, the host cell can provide for production of FRS containing polypeptide (e.g., due to lack of expression of an FGE that facilitates production of the aldehyde tag), which then would be modified by exposure to a FGE.

In general, the polypeptides described herein may be expressed in prokaryotes or eukaryotes in accordance with conventional ways, depending upon the purpose for expression. A host cell, e.g., a genetically modified host cell, that comprises a nucleic acid encoding a target polypeptide can further optionally comprise a recombinant FGE, which may be endogenous or heterologous to the host cell.

Host cells for production (including large scale production) of an unconverted or (where the host cell expresses a suitable FGE) converted FRS containing polypeptide, or for production of an FGE (e.g., for use in a cell-free method) can be selected from any of a variety of available host cells. Exemplary host cells include those of a prokaryotic or eukaryotic unicellular organism, such as bacteria (e.g., Escherichia coli strains, Bacillus spp. (e.g., B. subtilis), and the like) yeast or fungi (e.g., S. cerevisiae, Pichia spp., and the like), and other such host cells can be used. Exemplary host cells originally derived from a higher organism such as insects, vertebrates, particularly mammals, (e.g. CHO, HEK, and the like), may be used as the expression host cells.

Specific expression systems of interest include bacterial, yeast, insect cell and mammalian cell derived expression systems.

### Methods for Production and Conjugation of an Aldehyde Tag

Production of an aldehyde tag in FRS containing polypeptide can be accomplished by cell-based (in vivo) or cell-free methods (in vitro). Similarly, conjugation of an aldehyde tag in a polypeptide can be accomplished by cell-based (in vivo) or cell-free methods (in vitro). These are described in more detail below.

### “In Vivo” Host Cells Production and Conjugation

Production of an aldehyde tag in an FRS polypeptide can be accomplished by expression of the FRS-containing polypeptide in a cell that contains a suitable FGE. In this embodiment, conversion of the cysteine or serine to produce the aldehyde tag occurs during or following translation in the host cell.

Depending on the nature of the target polypeptide containing the FRS, following production of the aldehyde tag, the polypeptide is either retained in the host cell intracellularly, is secreted, or is associated with the host cell extracellular membrane. Where the FRS-containing polypeptide is present at the cell surface, conjugation of the produced aldehyde tag can be accomplished by use of a reactive partner to attach a moiety of the reactive partner to an fGly residue of a surface accessible aldehyde tag under physiological conditions. Conditions suitable for use to accomplish conjugation of a reactive partner moiety to an aldehyde tagged polypeptide are similar to those described in Mahal et al. (1997 May 16) Science 276(5315):1125-8.

The host cells used to produce proteins for the methods of this invention may be cultured in a variety of media. Commercially available growth media such as Ham's F10 (Sigma), Minimal Essential Medium (MEM), (Sigma), RPMI-1640 (Sigma), and Dulbecco's Modified Eagle's Medium ((DMEM), (Sigma), Expi293 media, etc., are suitable for culturing the host cells. In addition, any of the media described in Ham et al (1979) Meth. Enz. 58:44, Barnes et al (1980) Anal. Biochem. 102:255, U.S. Pat. Nos. 4,767,704; 4,657,866; 4,927,762; 4,560,655; 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as MES and HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

In certain embodiments, where the present method is carried out in cells, the cells are in vitro, e.g., in in vitro cell culture, e.g., where the cells are cultured in vitro in a single-cell suspension or as an adherent cell culture. In some embodiments, the cells are cultured in the presence of an oxidation reagent that can activate FGE. The oxidation reagent may be Cu²⁺. In some embodiments, a cell expressing an FGE is cultured in the presence of a suitable amount of Cu²⁺ in the culture medium. In certain aspects, the Cu²⁺ is present in the cell culture medium at a concentration of from 1 nM to 100 mM, such as from 0.1 μM to 10 mM, from 1 μM to 1 mM, from 2 μM to 500 μM, from 4 μM to 300 μM, or from 5 μM to 200 μM (e.g., from 10 μM to 150 μM). The culture medium may be supplemented with any suitable copper salt to provide for the Cu²⁺. Suitable copper salts include, but are not limited to, copper sulfate (i.e., copper(II) sulfate, CuSO₄), copper citrate, copper tartrate, copper nitrate, and any combination thereof.

### “In Vitro” (Cell-Free) Conversion and Conjugation

In vitro (cell-free) production of an aldehyde tag in an FRS-containing polypeptide can be accomplished by contacting the polypeptide with an FGE under conditions suitable for conversion of a cysteine or serine of a sulfatase motif to an fGly. For example, nucleic acid encoding an FRS-containing polypeptide can be expression in an in vitro transcription/translation system in the presence of a suitable FGE to provide for production of aldehyde tagged polypeptides.

Alternatively, an FRS-containing polypeptide can be isolated following recombinant production in a host cell lacking a suitable FGE or by synthetic production. The isolated an FRS-containing polypeptide is then contacted with a suitable FGE under conditions to provide for aldehyde tag production.

With respect to conjugation of an aldehyde tag, conjugation is normally carried out in vitro. Aldehyde tagged polypeptide is isolated from a production source (e.g., recombinant host cell production, synthetic production), and contacted with a reactive partner under conditions suitable to provide for conjugation of a moiety of the reactive partner to the fGly of the aldehyde tag. If the aldehyde tag is not solvent accessible, the aldehyde tagged polypeptide can be unfolded by methods known in the art prior to reaction with a reactive partner.

### EXEMPLARY NON-LIMITING ASPECTS OF THE DISCLOSURE

Aspects, including embodiments, of the present subject matter described above may be beneficial alone or in combination with one or more other aspects or embodiments. Without limiting the foregoing description, certain non-limiting aspects of the disclosure are provided below as separately numbered clauses. As will be apparent to those of ordinary skill in the art upon reading this disclosure, each of the individually numbered clauses may be used or combined with any of the preceding or following individually numbered clauses. This is intended to provide support for all such combinations of aspects. It will be apparent to one of ordinary skill in the art that various changes and modifications can be made without departing from the spirit or scope of the invention.

Such clauses may include:
1. A method of reducing cleavage of a protein comprising a formylglycine (fGly) amino acid, the method comprising:
protecting the protein from exposure to visible light having a wavelength of 500 nm or lower.
2. The method of clause 1, wherein the method comprises culturing a cell, wherein the cell comprises the protein and wherein protecting the protein from exposure to visible light having a wavelength of 500 nm or lower comprises using visible light having a wavelength higher than 500 nm during the culturing.
3. The method of clause 1, comprising synthesizing the protein, wherein protecting the protein from exposure to visible light having a wavelength of 500 nm or lower comprises synthesizing the protein in visible light having a wavelength higher than 500 nm.
4. The method of clause 1, comprising purifying the protein, wherein protecting the protein from exposure to visible light having a wavelength of 500 nm or lower comprises purifying the protein in visible light having a wavelength higher than 500 nm.
5. The method of clause 4, wherein purifying comprises isolating the protein from a cell or a cell culture medium comprising the cell.
6. The method of any one of clauses 2-5, wherein the visible light having a wavelength higher than 500 nm comprises a wavelength higher than 500 nm and lower than 620 nm.
7. The method of any one of clauses 2-6, wherein the visible light having a wavelength higher than 500 nm is generated by a light source that produces visible light limited to green light, yellow light and/or orange light.
8. The method of any one of clauses 2-7, wherein the visible light having a wavelength higher than 500 nm is generated by passing visible light through a filter that significantly blocks transmission of visible light in the range of 380 nm to 500 nm.
9. The method of clause 1, wherein the method comprises culturing a cell, wherein the cell comprises the protein and protecting the protein from exposure to visible light having a wavelength of 500 nm or lower comprises culturing the cell in absence of visible light.
10. The method of clause 1, comprising synthesizing the protein, wherein protecting the protein from exposure to visible light having a wavelength of 500 nm or lower comprises synthesizing the protein in absence of visible light.
11. The method of any one of clauses 1-10, wherein the cleavage of the protein occurs at or adjacent the fGly amino acid.
12. The method of any one of clauses 1-11, wherein the fGly amino acid is generated in a formylglycine-generating enzyme (FGE) recognition site.
13. The method of clause 12, wherein the FGE recognition site comprises the consensus sequence X₁C/SX₂P/AX₃R, wherein X₁ is present or absent and, when present, is any amino acid, with the proviso that when the FGE recognition site is at an N-terminus of the protein, X₁ is present; and X₂ and X₃ are each independently any amino acid.
14. The method of clause 13, wherein the FGE recognition site comprises the sequence LCTPSR.
15. The method of clause 13, wherein the FGE recognition site comprises the consensus sequence X₁SX₂PX₃R.
16. The method of clause 15, wherein the FGE recognition site comprises the sequence LSTPSR.
17. The method of clause 13, wherein the FGE recognition site comprises the consensus sequence X₁CX₂AX₃R.
18. The method of clause 17, wherein the FGE recognition site comprises the sequence LCTASR.
19. The method of clause 13, wherein the FGE recognition site comprises the sequence LCTASA.
20. The method of any one of clauses 1-19, wherein the protein is an antibody and/or a therapeutic protein.
21. The method of any one of clauses 1-20, wherein cleavage of the protein occurs in the presence of a molecule that is photoactivated to release singlet oxygen species.
22. The method of clause 21, wherein the molecule is photoactivated by exposure to visible light having a wavelength of 500 nm or lower.
23. The method of any one of clauses 1-20, wherein cleavage of the protein occurs in the presence of a flavin.
24. The method of clause 23, wherein the flavin is riboflavin.
25. The method of clause 23, wherein the flavin is flavin mononucleotide or flavin adenine dinucleotide.
26. A method of inducing cleavage of a protein in a target region, the target region comprising a formylglycine (fGly) amino acid, the method comprising:
exposing the protein to light comprising a wavelength of 300 nm-500 nm.
27. The method of clause 26, wherein the light is limited to a wavelength between 325 nm-495 nm.
28. The method of clause 26 or 27, wherein the method comprises introducing the fGly amino acid at the target region.
29. The method of any one of clauses 26-28, wherein exposing the protein to the light comprises:
culturing a cell comprising the protein in the light; and/or
purifying the protein in the light.
30. The method of any one of clauses 26-29, wherein the cleavage of the protein occurs at or adjacent the fGly amino acid.
31. The method of any one of clauses 26-30, wherein the fGly amino acid is generated in a formylglycine-generating enzyme (FGE) recognition site.
32. The method of clause 31, wherein the FGE recognition site comprises the consensus sequence X₁C/SX₂P/AX₃R, wherein X₁ is present or absent and, when present, is any amino acid, with the proviso that when the FGE recognition site is at an N-terminus of the protein, X₁ is present; and X₂ and X₃ are each independently any amino acid.
33. The method of clause 32, wherein the FGE recognition site comprises the sequence LCTPSR.
34. The method of clause 32, wherein the FGE recognition site comprises the consensus sequence X₁SX₂PX₃R.
35. The method of clause 34, wherein the FGE recognition site comprises the sequence LSTPSR.
36. The method of clause 32, wherein the FGE recognition site comprises the consensus sequence X₁CX₂AX₃R.
37. The method of clause 36, wherein the FGE recognition site comprises the sequence LCTASR.
38. The method of clause 32, wherein the FGE recognition site comprises the sequence LCTASA.
39. The method of any one of clause 26-38, wherein the method comprises introducing a formylglycine-generating enzyme (FGE) recognition site at the target region.
40. The method of any one of clauses 26-39, wherein the protein is an antibody comprising an Fc region and the target region is located between the Fc region and a CH1 domain of the antibody.
41. The method of any one of clauses 26-39, wherein the protein comprises a purification tag and wherein the target region is located between the protein sequence and the purification tag.
42. The method of any one of clauses 26-41, comprising exposing the protein to light comprising a wavelength of 300 nm-500 nm in the presence of a molecule that is photoactivated to release singlet oxygen species.
43. The method of clause 42, wherein the molecule is photoactivated by exposure to visible light having a wavelength of 500 nm or lower.
44. The method of any one of clauses 26-41, comprising exposing the protein to light comprising a wavelength of 300 nm-500 nm in the presence of a flavin.
45. The method of clause 44, wherein the flavin is riboflavin.
46. The method of clause 44, wherein the flavin is flavin mononucleotide or flavin adenine dinucleotide.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1: Photo-Clipping of fGly-Containing Proteins and Peptides

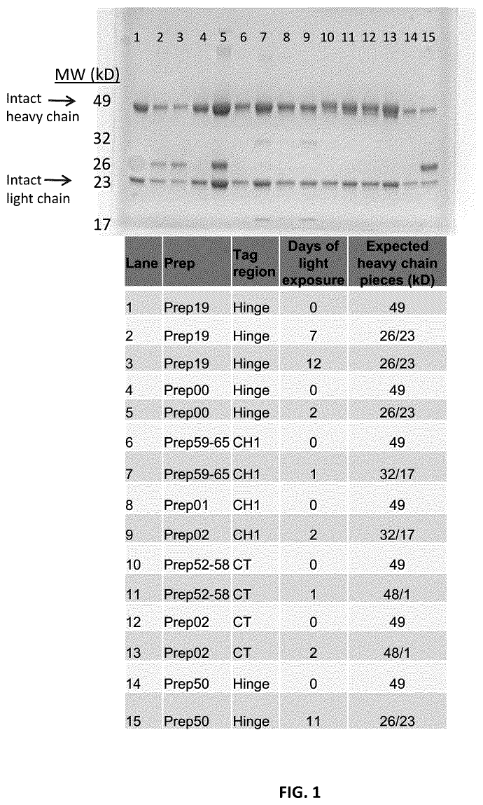

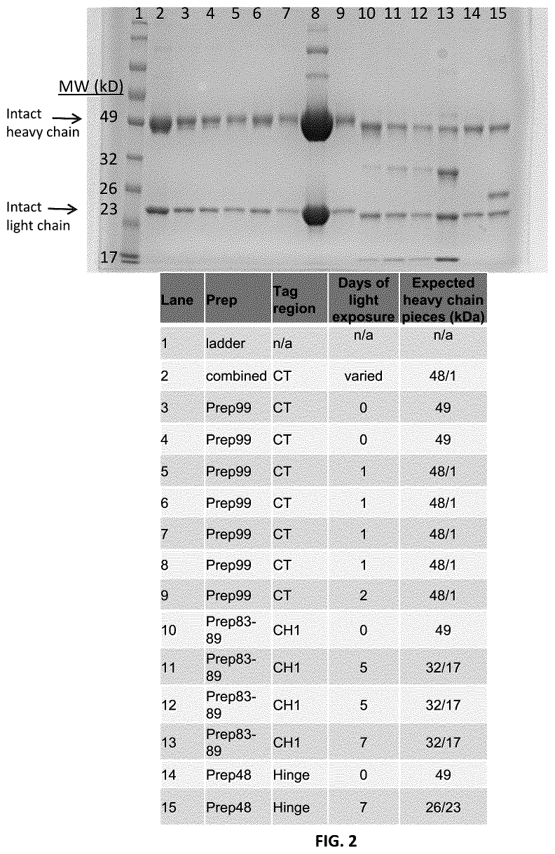

SDS-PAGE of aldehyde-tagged antibody preparations (preps) where an antibody batch was purified from conditioned media at two different times, early and late, relative to when harvest of media from cell culture was performed. See FIGS. 1 and 2. LC/MS analysis of aldehyde-tagged antibodies purified early or late from antibody production batches was performed. Comparative results obtained from early and late antibody preps reveal new peaks observed in the late preps that roughly align by mass to potential fragmentation occurring at the aldehyde tag between the fGly and Thr residues in the formylglycine-generating enzyme (FGE) recognition site LCTPSR (SEQ ID NO:3) present in these antibodies.

Assessment of the stability of an fGly-containing peptide in cell culture media (Epi293 media) at 4° C. or 37° C. was performed. The fGly peptide was undetectable after a day at 4° C. in a deli case (glass door, facing a window). The fGly peptide is unchanged in concentration after a day at 37° C. in a closed oven which allowed no visible light.

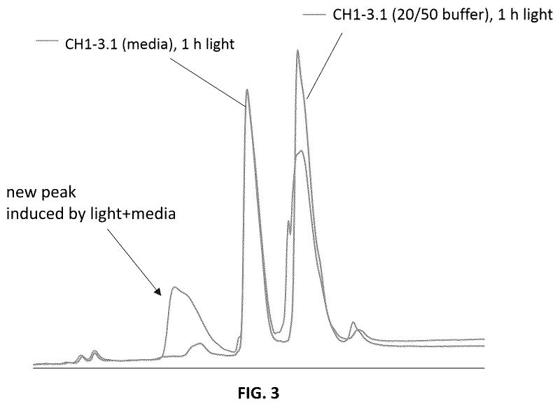

An fGly-containing mAb either in cell culture media or in 20 mM sodium citrate, 50 mM sodium chloride was exposed for 1 h to light from a desk lamp and then analyzed by HPLC. See FIG. 3.

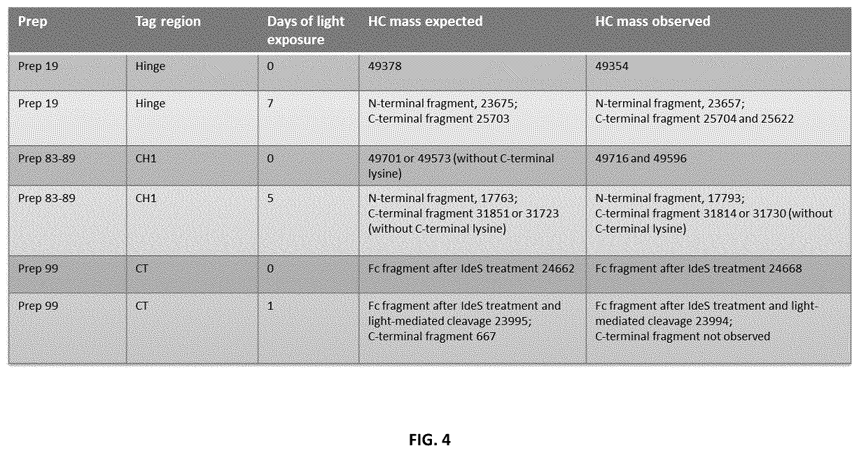

FIG. 4. Mass spectrometric analysis of antibody fragments in fGly-containing protein preparations before and after exposure to light. Antibodies were deglycosylated using PNGaseF and analyzed by RP-LC/MS on an ABSciex 4000 QTRAP instrument. Preps 19 and 83-89 were reduced with DTT prior to analysis. Prep 99 was treated with IdeS protease (Promega) to liberate the Fc from the F(ab)² domain.

### Antibody Sequences:

Antibody heavy chain constant regions bearing FGE recognition sites in different locations. The FGE recognition site, LCTPSR, is shown in bold text. After conversion by FGE to LfGlyTPSR, the protein was cleaved between the leucine and fGly residues upon exposure to light in the presence of riboflavin.

Hinge 1.6:
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSCDGLCTPSRTHTCPPCPAPELLGGPSVFLFPPK
PKDTLMISRTPEVTCVVVDVSHEDPEVKFNWYVDGVEVHNAKTKP
REEQYNSTYRVVSVLTVLHQDWLNGKEYKCKVSNKALPAPIEKTI
SKAKGQPREPQVYTLPPSREEMTKNQVSLTCLVKGFYPSDIAVEW
ESNGQPENNYKTTPPVLDSDGSFFLYSKLTVDKSRWQQGNVFSCS
VMHEALHNHYTQKSLSLSPGK
CH1-3.1:
ASTKGPSVFPLAPSSKSTSGGTAALGCLVKDYFPEPVTVSWNSGA
LCTPSRGVHTFPAVLQSSGLYSLSSVVTVPSSSLGTQTYICNVNH
KPSNTKVDKKVEPKSCDKTHTCPPCPAPELLGGPSVFLFPPKPKD
TLMISRTPEVTCVVVDVSHEDPEVKFNWYVDGVEVHNAKTKPREE
QYNSTYRVVSVLTVLHQDWLNGKEYKCKVSNKALPAPIEKTISKA
KGQPREPQVYTLPPSREEMTKNQVSLTCLVKGFYPSDIAVEWESN
GQPENNYKTTPPVLDSDGSFFLYSKLTVDKSRWQQGNVFSCSVMH
EALHNHYTQKSLSLSPGK
CT:
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSCDKTHTCPPCPAPELLGGPSVFLFPPKPKDTLM
ISRTPEVTCVVVDVSHEDPEVKFNWYVDGVEVHNAKTKPREEQYN
STYRVVSVLTVLHQDWLNGKEYKCKVSNKALPAPIEKTISKAKGQ
PREPQVYTLPPSREEMTKNQVSLTCLVKGFYPSDIAVEWESNGQP
ENNYKTTPPVLDSDGSFFLYSKLTVDKSRWQQGNVFSCSVMHEAL
HNHYTQKSLSLSPGSLCTPSRGS

FIG. 1. SDS-PAGE of aldehyde-tagged antibodies purified from conditioned media before or after exposure to light. Antibodies were reduced with DTT prior to analysis.

FIG. 2. SDS-PAGE of aldehyde-tagged antibodies purified from conditioned media before or after exposure to light. Antibodies were reduced with DTT prior to analysis.

FIG. 3. Incubation of an fGly-containing antibody in cell culture media with light results in a cleavage product. An fGly-containing antibody bearing the aldehyde tag at the CH1-3.1 position was incubated with light in either Expi293 cell culture media or in 20 mM sodium citrate, 50 mM sodium chloride, pH 5.5. The samples were exposed for 1 h to light from a desk lamp and then analyzed by C8 reversed phase HPLC. Samples were prepared for HPLC analysis by the addition of 50 mM DTT and 0.5% SDS (final concentrations) and heating at 50° C. for 30 min.

FIG. 4. Mass spectrometric analysis of antibody fragments in fGly-containing protein preparations before and after exposure to light. Antibodies were deglycosylated using PNGaseF and analyzed by RP-LC/MS on an ABSciex 4000 QTRAP instrument. Preps 19 and 83-89 were reduced with DTT prior to analysis. Prep 99 was treated with IdeS protease (Promega) to liberate the Fc from the F(ab)² domain.

### Example 2: Photo-Clipping of fGly-Containing Proteins and Peptides is Mediated by Riboflavin

While determining analytical conditions for monitoring fGly-containing peptide degradation, an unexpected peak was detected by UV/Vis in the cell culture media. The peak was postulated to indicate presence of Vitamin B12. Vitamin B12 can perform a variety of photochemical reactions to either sensitize other molecules or generate singlet oxygen, which can react with ground state singlet organic molecules.

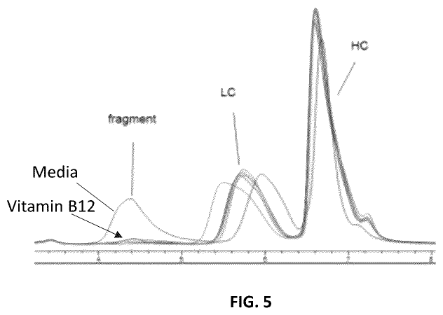

Effect of Vitamin B12 on fGly-containing peptide fragmentation was tested. Significant fragment peak only seen when mAb was incubated with media. Addition of up to 75 molar equivalents of Vitamin B12 only induced a minor amount of photofragmentation. See FIG. 5.

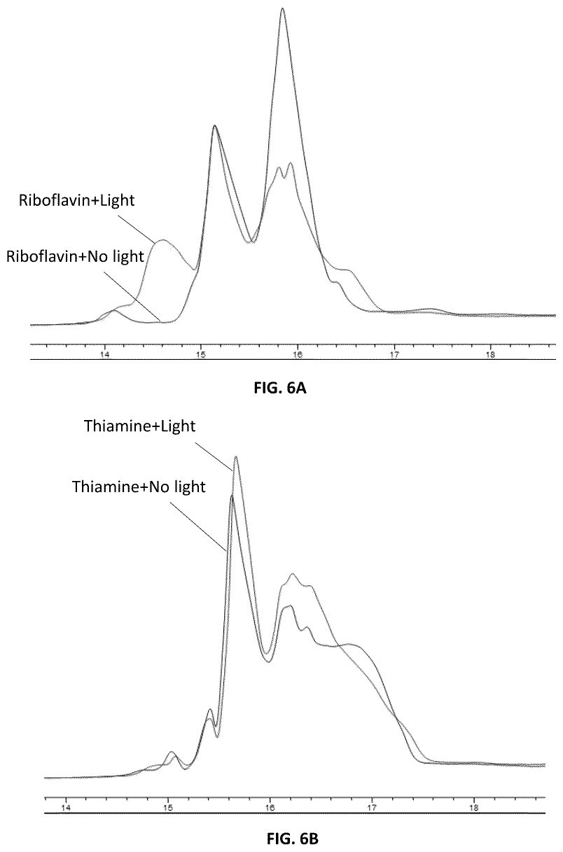

Effect of other light-absorbing molecules, such as, riboflavin and thiamine, found in cell culture media on fGly-containing CH1-aldehyde tagged antibody fragmentation was tested. Riboflavin+light induced a new protein fragment (FIG. 6A), but thiamine did not appear to have a significant effect on protein cleavage (FIG. 6B).

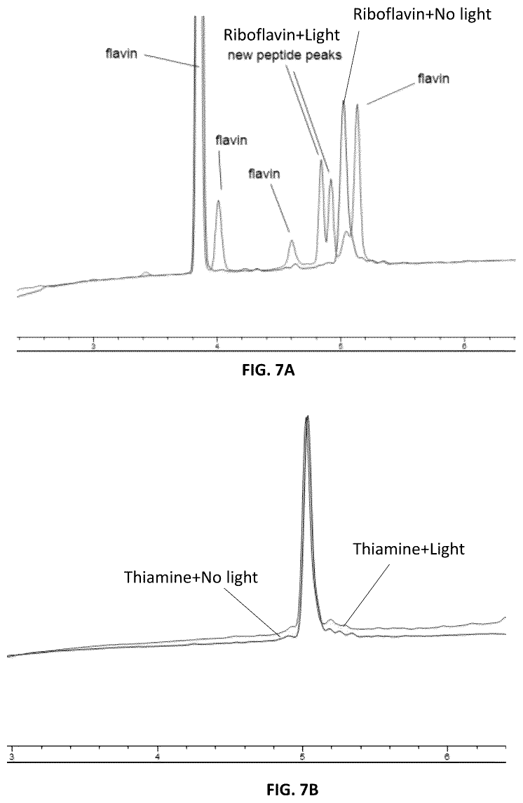

Effect of riboflavin and thiamine on fGly containing peptide (“fGly peptide,” sequence: ALfGlyTPSRGSLFTGR (SEQ ID NO:1)) was tested. New peptide peaks were detected by LCMS analysis of fGly peptide incubated with Riboflavin+light (FIG. 7A). Cleavage of the fGly peptide incubated with thiamine+light was not detected (FIG. 7B).

LC/MS analysis of the fGly peptide sample after exposure to riboflavin and light revealed a new peptide fragment representing the C-terminal portion of the original peptide. The sequence of the observed peptide fragment is: fGlyTPSRGSLFTGR.

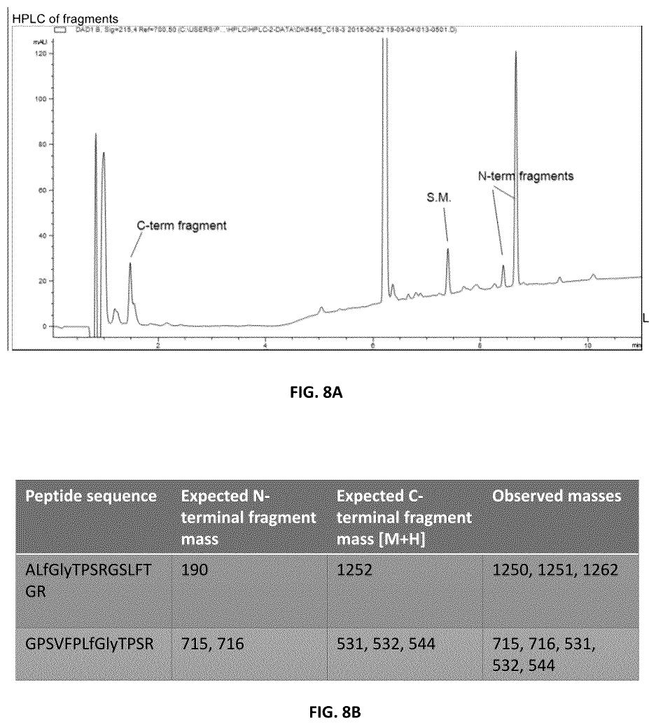

In order to detect the N-terminal side of a cleaved peptide, a different peptide sequence was selected with more amino acids preceding the LfGlyTPSR sequence. This peptide, GPSVFPLfGlyTPSR (SEQ ID NO:2), was incubated at 1 mg/mL with 50 mg/L of riboflavin illuminated by a desk lamp for 30 min. Then, the sample was analyzed by C18 reverse phase chromatography. Both N- and C-terminal fragments were observed. FIGS. 8A and 8B. S.M., starting material. Both the N-fragment (GPSVFPL) and the C-term fragment (fGlyTPSR) were observed (FIG. 8A). Results from mass spectrometric analysis of peptide fragments observed after cleavage with riboflavin and light is tabulated in FIG. 8B. The expected N-terminal fragment of the ALfGlyTPSRGSLFTGR (SEQ ID NO:1) peptide was AL. This dipeptide was not observed by chromatography or by mass spectrometry as it was likely not retained on the column under the experimental conditions. The GPSVFPLfGlyTPSR (SEQ ID NO:2) peptide was used because it would generate a longer N-terminal fragment, which was detected both by chromatography (FIG. 8A) and by mass spectrometry (FIG. 8B).

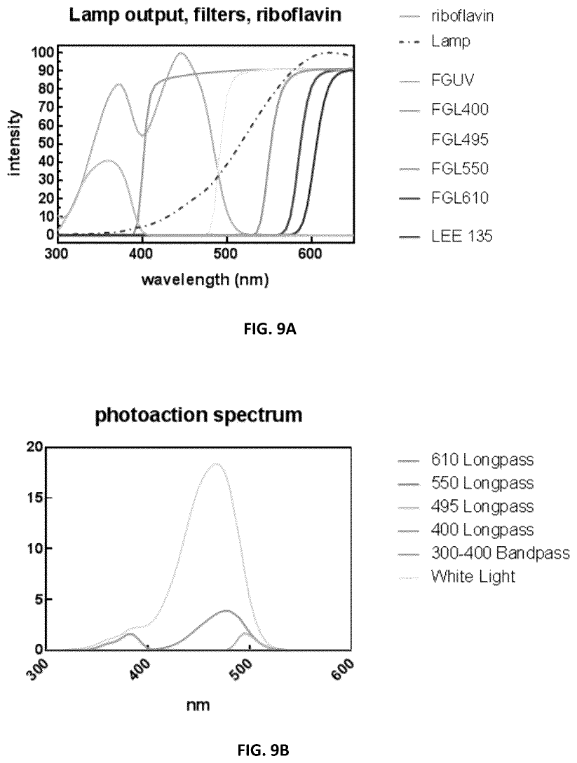

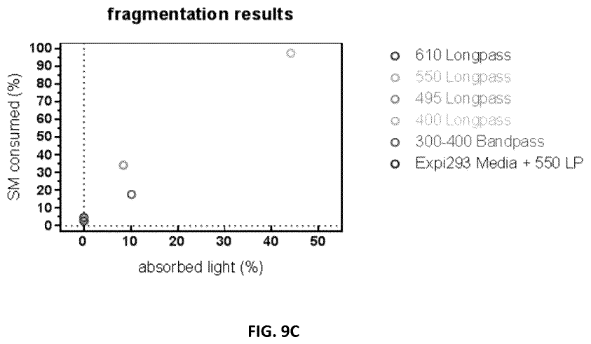

Effect of light having a wavelength that corresponds to the absorption spectra of riboflavin was tested. The GPSVFPLfGlyTPSR (SEQ ID NO:2) peptide was incubated with riboflavin+light with or without a bandpass filter and analyzed by HPLC. No fragmentation was observed when using a longpass filter of 550 or greater (FIG. 9C). FIG. 9A shows the instrumentation used for determining effect of wavelengths of visible light on riboflavin mediated cleavage of GPSVFPLfGlyTPSR (SEQ ID NO:2) peptide. Lamp output of a broad-spectrum lamp (QTH10) from Thor labs is plotted in FIG. 9A. Wavelengths of light permitted through the listed filters are shown. Wavelengths of light absorbed by riboflavin are also plotted. Riboflavin absorbs light in the range of about 300 nm-500 nm with peak absorption at about 450 nm and a lower peak at about 380 nm. Photoaction, FIG. 9B. White light and all filters that allowed light to pass in the window of riboflavin absorbance (<300-500 nm) were associated with cleavage. Fragmentation results, FIG. 9C. Of the filtered light, the 400 nm longpass filter was associated with the highest level of cleavage (˜95% of starting material consumed). This corresponded to the area of peak riboflavin light absorption at around 450 nm. No fragmentation was observed when using a longpass filter of 550 or greater. Thus, riboflavin and light-mediated cleavage of fGly-containing peptides was dependent on the absorption of light by riboflavin. Blocking that absorption effectively protected the peptide from cleavage.

The mechanism of photo-clipping was further explored by adding a singlet oxygen quencher (azide) to the reaction. Addition of azide inhibited photo-clipping induced by light and riboflavin on the fGly-containing peptide, indicating that a significant portion of the fragmentation results from fGly reacting with singlet oxygen.

The foregoing experiments establish that in the presence of light and riboflavin, fGly containing proteins are cleaved. The cleavage appears to occur between fGly and the amino acid immediately N-terminal to fGly. This cleavage is prevented when the light to which the protein is exposed does not include the wavelengths that are absorbed by riboflavin. In other words, preventing exposure of fGly containing proteins to wavelengths in the range of about 300-500 nm prevents photo-cleavage of the proteins even in the presence of riboflavin, e.g., riboflavin present in the cell culture medium.

Thus, the data presented here indicate that when an fGly containing protein is present in a solution that also has riboflavin, exposure of the protein to light in the range of 500 nm or lower should be limited to reduce photo-cleavage. In cases where light is needed to process the protein, e.g., purify the protein, light limited to a wavelength of higher than 500 nm, e.g. blue light, green light, yellow light, red light and/or orange light can be used for providing visibility while protecting the protein from degradation till riboflavin is removed from the solution in which the protein is present.

This data also shows that where cleavage of a protein at a target site is desired, light in the wavelength that does cause photo-clipping and inclusion of riboflavin in the solution in which the protein is present can be used to achieve cleavage at the target site, by, e.g., including an fGly residue in the target site. This cleavage can be enhanced by exposing the protein to a higher intensity of light in the wavelength range absorbed by riboflavin, e.g., in the range of about 300-500 nm.

The inhibition of photo-clipping by azide indicates that a significant portion of the fragmentation results from fGly reacting with singlet oxygen. Since other flavins, e.g., flavin mononucleotide (FMN) and flavin adenine dinucleotide (FAD) are known to generate reactive oxygen (ROS) upon exposure to light, it is expected that flavins such as riboflavin analogs and derivatives which generate singlet oxygen upon exposure to light (e.g., light in range of about 300-500 nm) can be used for inducing cleavage of a protein in a target region which target region includes an fGly amino acid. Furthermore, it is expected that flavin-mediated cleavage of a protein comprising an fGly amino acid can be reduced by protecting the protein from exposure to visible light absorbed by the flavin to release singlet oxygen, e.g., by protecting the protein from exposure to visible light having a wavelength of 500 nm or lower.

FIG. 5. Testing the effect of vitamin B12 and light on fragmentation of an fGly-containing peptide. An fGly-containing antibody bearing the aldehyde tag at the CH1-3.1 position was incubated at 10 μM in buffer with increasing molar equivalents of vitamin B12. Specifically, vitamin B12 was tested at molar equivalents of 1, 10, 25, 50, and 75 relative to antibody. As a positive control for protein cleavage, antibody was incubated in Expi293 media. Samples were exposed to light from a desk lamp for 1 h and then were analyzed by C8 reverse phase chromatography. Samples were prepared for HPLC analysis by the addition of 50 mM DTT and 0.5% SDS (final concentrations) and heating at 50° C. for 30 min. Significant cleavage was only seen when the antibody was incubated with media (top panel). Adding up to 75 molar equivalents of vitamin B12 only induced a minor amount of photofragmentation (data not shown).

FIGS. 6A and 6B. Assessing the potential of other light-absorbing molecules found in cell culture media to mediate cleavage of an fGly-containing protein. An fGly-containing antibody bearing the aldehyde tag at the CH1-3.1 position was incubated at 1 mg/mL in buffer with either 120 μM riboflavin or 1 mM thiamine. Samples were exposed to light from a desk lamp for 1 h and then were buffer exchanged into 0.1 M ammonium bicarbonate buffer using 30 MWCO filters. Samples were disassembled by the addition of 50 mM DTT and 0.5% SDS (final concentrations) and heating at 50° C. for 30 min. Then, samples were analyzed by C8 reverse phase chromatography. Riboflavin and light induced a new protein fragment (FIG. 6A), but thiamine did not appear to do this (FIG. 6B).

FIGS. 7A and 7B. Testing the effect of thiamine or riboflavin on the fGly-containing peptide, ALfGlyTPSRGSLFTGR (SEQ ID NO:1). Peptide in buffer was mixed with either riboflavin (FIG. 7A) or thiamine (FIG. 7B) and exposed to light from a desk lamp for 1 h followed by analysis by C18 reversed phase chromatography. Riboflavin and light, but not thiamine and light, induced new peptide peaks.

FIG. 8A. Riboflavin and light-mediated cleavage of the GPSVFPLfGlyTPSR (SEQ ID NO:2) peptide yields N- and C-terminal fragments that are detected by reverse phase chromatography. The peptide GPSVFPLfGlyTPSR (SEQ ID NO:2) was incubated at 1 mg/mL with 50 mg/L of riboflavin illuminated by a desk lamp for 30 min. Then, the sample was analyzed by C18 reverse phase chromatography. Both N- and C-terminal fragments were observed. S.M., starting material.

FIG. 8B. Mass spectrometric analysis of peptide fragments observed after cleavage with riboflavin and light.

FIG. 9A. Relevant UV-Vis range covered by the lamp output, riboflavin absorption, and filters. A ThorLabs broad spectrum lamp (QTH10) was used as the light source. The ThorLabs filter set (UV to NIR) was tested.

FIGS. 9B-9C. Assessing the effect of light wavelength on riboflavin-mediated peptide cleavage. The GPSVFPLfGlyTPSR (SEQ ID NO:2) peptide was incubated at 20 μM in triethanolamine buffer pH 7.4 with 100 μM riboflavin. Samples were exposed to light from a ThorLabs broad spectrum lamp (QTH10) for 1 h at room temperature. For some samples, the light source was covered with a filter from ThorLabs UV-NIR filter set. After incubation, samples were analyzed by C18 reverse phase chromatography and cleavage was quantified by monitoring loss of starting material (SM).

### Example 3. Kinetics of Riboflavin-Mediated Cleavage of fGly-Containing Proteins

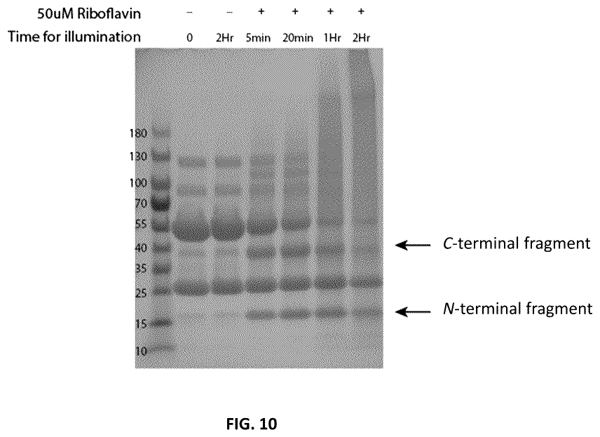

An fGly-containing antibody bearing the aldehyde tag at the CH1-3.1 position was incubated at 5 μM in buffer with or without 50 μM riboflavin. The sample was exposed to light for varying lengths of time ranging from 5 min to 2 hours. Then, the material was reduced with DTT and analyzed by SDS-PAGE to detect starting material (light chain and heavy chain at 23 and 49 kD, respectively) and heavy chain cleavage products (N-terminal and C-terminal fragments at 17 and 32 kD, respectively). See FIG. 10. Note that samples were not deglycosylated, increasing the apparent molecular weight of the analytes.

### Example 4. Effect of the Ratio of Riboflavin to fGly-Containing Protein on Cleavage

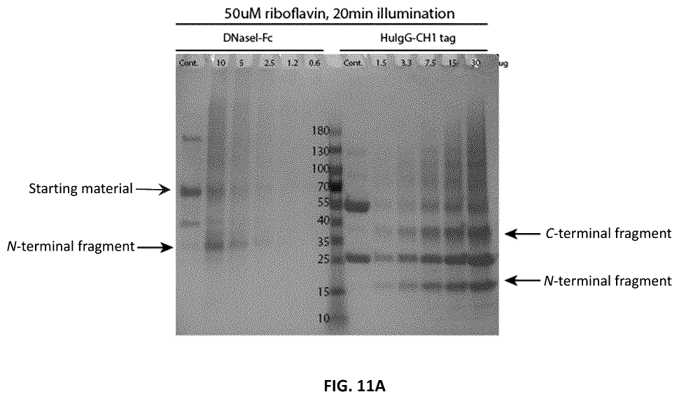

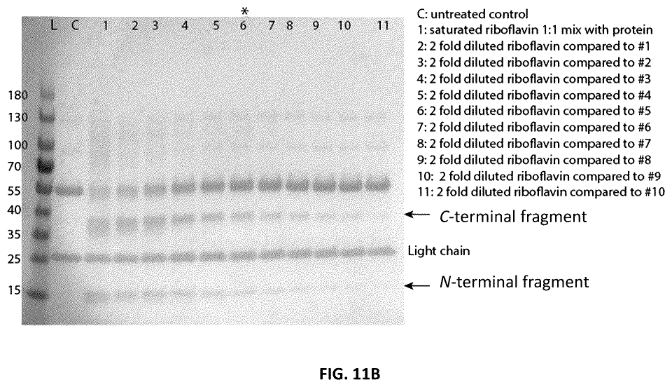

Two fGly-containing protein substrates were tested. One was human DNAaseI appended to an Fc domain bearing the aldehyde tag at the enzyme-Fc junction (DNAseI-Fc). The other was an fGly-containing antibody bearing the aldehyde tag at the CH1-3.1 position (HuIgG-CH1 tag). Varying amounts of protein (as shown in FIG. 11A) were incubated in 10 μL buffer containing 50 μM riboflavin. Samples were exposed to light for 20 min. Then, the material was reduced with DTT and analyzed by SDS-PAGE to detect starting material and cleavage products. For DNAseI-Fc, starting material was 55 kD and cleavage products were 29 and 26 kD for N-terminal and C-terminal fragments, respectively. For HuIgG-CH1 tag, starting material was 23 and 49 kD for antibody light and heavy chains, respectively. HuIgG-CH1 tag heavy chain cleavage products were 17 and 32 kD for N-terminal and C-terminal fragments, respectively. Note that samples were not deglycosylated, increasing the apparent molecular weight of the analytes.

An fGly-containing antibody bearing the aldehyde tag at the CH1-3.1 position was incubated at 1 μM with varying concentrations of riboflavin ranging from 60 μM (50% of a saturated solution, sample #1) to 50 nM (sample #11). See FIG. 11B. Samples were exposed to light for 20 min. Then, the material was reduced with DTT and analyzed by SDS-PAGE to detect starting material and cleavage products. The optimal riboflavin to protein ratio was observed in sample #6, at approximately 1.6 μM riboflavin to 1 μM antibody. Starting material was 23 and 49 kD, representing antibody light and heavy chains, respectively. Antibody heavy chain cleavage products were 17 and 32 kD representing N-terminal and C-terminal fragments, respectively. Note that samples were not deglycosylated, increasing the apparent molecular weight of the analytes.

Human DNAseI-Fc construct sequence, bearing the aldehyde tag at the enzyme-Fc junction. The FRS, LCTPSR, is shown in bold text.

DNaseI-Fc:
LKIAAFNIQTFGETKMSNATLVSYIVQILSRYDIALVQEVRDSHL
TAVGKLLDNLNQDAPDTYHYVVSEPLGRNSYKERYLFVYRPDQVS
AVDSYYYDDGCEPCGNDTFNREPAIVRFFSRFTEVREFAIVPLHA
APGDAVAEIDALYDVYLDVQEKWGLEDVMLMGDFNAGCSYVRPSQ
WSSIRLWTSPTFQWLIPDSADTTATPTHCAYDRIVVAGMLLRGAV
VPDSALPFNFQAAYGLSDQLAQAISDHYPVEVMLCTPSRSGGGGS
DKTHTCPPCPAPELLGGPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
EEMTKNQVSLTCLVKGFYPSDIAVEWESNGQPENNYKTTPPVLDS
DGSFFLYSKLTVDKSRWQQGNVFSCSVMHEALHNHYTQKSLSLSP
GK.

1. A method of reducing cleavage of a protein comprising a formylglycine (fGly) amino acid, the method comprising:
protecting the protein from exposure to visible light having a wavelength of 500 nm or lower.

2. The method of claim 1, wherein the method comprises culturing a cell, wherein the cell comprises the protein and wherein protecting the protein from exposure to visible light having a wavelength of 500 nm or lower comprises using visible light having a wavelength higher than 500 nm during the culturing.

3. The method of claim 1, comprising synthesizing the protein, wherein protecting the protein from exposure to visible light having a wavelength of 500 nm or lower comprises synthesizing the protein in visible light having a wavelength higher than 500 nm.

4. The method of claim 1, comprising purifying the protein, wherein protecting the protein from exposure to visible light having a wavelength of 500 nm or lower comprises purifying the protein in visible light having a wavelength higher than 500 nm.

5. The method of claim 4, wherein purifying comprises isolating the protein from a cell or a cell culture medium comprising the cell.

6. The method of claim 2, wherein the visible light having a wavelength higher than 500 nm comprises a wavelength higher than 500 nm and lower than 620 nm.

7. The method of claim 2, wherein the visible light having a wavelength higher than 500 nm is generated by a light source that produces visible light limited to green light, yellow light and/or orange light.

8. The method of claim 2, wherein the visible light having a wavelength higher than 500 nm is generated by passing visible light through a filter that significantly blocks transmission of visible light in the range of 380 nm to 500 nm.

9. The method of claim 1, wherein the method comprises culturing a cell, wherein the cell comprises the protein and protecting the protein from exposure to visible light having a wavelength of 500 nm or lower comprises culturing the cell in absence of visible light.

10. The method of claim 1, comprising synthesizing the protein, wherein protecting the protein from exposure to visible light having a wavelength of 500 nm or lower comprises synthesizing the protein in absence of visible light.

11. The method of claim 1, wherein the cleavage of the protein occurs at or adjacent the fGly amino acid.

12. The method of claim 1, wherein the fGly amino acid is generated in a formylglycine-generating enzyme (FGE) recognition site.

13. The method of claim 12, wherein the FGE recognition site comprises the consensus sequence X₁C/SX₂P/AX₃R, wherein X₁ is present or absent and, when present, is any amino acid, with the proviso that when the FGE recognition site is at an N-terminus of the protein, X₁ is present; and X₂ and X₃ are each independently any amino acid.

14. The method of claim 13, wherein the FGE recognition site comprises the sequence LCTPSR.

15. The method of claim 13, wherein the FGE recognition site comprises the consensus sequence X₁SX₂PX₃R.

16. The method of claim 15, wherein the FGE recognition site comprises the sequence LSTPSR.

17. The method of claim 13, wherein the FGE recognition site comprises the consensus sequence X₁CX₂AX₃R.

18. The method of claim 17, wherein the FGE recognition site comprises the sequence LCTASR.

19. The method of claim 13, wherein the FGE recognition site comprises the sequence LCTASA.

20. The method of claim 1, wherein the protein is an antibody and/or a therapeutic protein.

21. The method of claim 1, wherein cleavage of the protein occurs in the presence of a molecule that is photoactivated to release singlet oxygen species.

22. The method of claim 21, wherein the molecule is photoactivated by exposure to visible light having a wavelength of 500 nm or lower.

23. The method of claim 1, wherein cleavage of the protein occurs in the presence of a flavin.

24. The method of claim 23, wherein the flavin is riboflavin.

25. The method of claim 23, wherein the flavin is flavin mononucleotide or flavin adenine dinucleotide.

26. A method of inducing cleavage of a protein in a target region, the target region comprising a formylglycine (fGly) amino acid, the method comprising:
exposing the protein to light comprising a wavelength of 300 nm-500 nm.

27.-46. (canceled)
